(19) United States
(12) Patent Application Publication
(10) Pub. No.: US 2023/0181303 A1
(43) Pub. Date: Jun. 15, 2023

(54) IMPLANT

(71) Applicant: OLYMPUS WINTER & IBE GMBH, Hamburg (DE)

(72) Inventors: Thorsten JÜRGENS, Hamburg (DE); Shun NOGUCHI, Machida-shi (JP)

(73) Assignee: OLYMPUS WINTER & IBE GMBH, Hamburg (DE)

(21) Appl. No.: 18/077,926

(22) Filed: Dec. 8, 2022

Related U.S. Application Data
(60) Provisional application No. 63/289,317, filed on Dec. 14, 2021.

(51) Int. Cl. A61F 2/04; A61M 29/02

(52) U.S. Cl. CPC A61F 2/04; A61M 29/02; A61F 2002/047; A61F 2210/0014; A61F 2210/0004

(57) ABSTRACT

An implant for efficient and gentle treatment of the urinary tract. This is achieved in that an implant has a pressure means structure, which is constructed from at least two pressure means. These pressure means are of rod-like design and exert a local ischemic pressure on the tissue of the urethra. In addition, the pressure means are secured with their distal ends on a common distal connecting body and with their proximal ends on a common proximal connecting body, wherein the distal connecting body is secured in a fixed manner to a tightening means and the proximal connecting body is movably mounted on the tightening means.

The invention relates to an implant in accordance with the preamble of claim 1.

Various methods and techniques are known for the treatment of the urinary tract, in particular benign prostatic enlargement (benign prostatic hyperplasia, BPH). In a minimally invasive, particularly gentle treatment of BPH symptoms, a removable implant is temporarily placed in the urethra or in the prostatic portion of the urethra of the patient. Such an implant is a wire structure made of a shape memory alloy, such as, for example, Nitinol. In a folded state, the wire structure is pushed into the correct position by a catheter in order to unfold there into its predetermined basic structure. This structure, which can be formed from three or four wires, is a basket structure. This basket structure widens the urethra. Owing to the expansion of the wire structure against the tissue of the urethra, the stricture tissue of the urethra is denatured over the course of a few days. This denaturation of the tissue takes place on account of the ischemic pressure of the individual wires on the cells of the tissue, leading to reduced or completely absent blood flow. As a result, the lack of blood supply leads to a lack of oxygen in the cells and ultimately to death of the cells. Within a few days, the tissue can be reduced to such an extent that the urinary flow almost normalizes. After completion of this treatment, the implant can be recovered from the urethra by means of a catheter.

With this known technique, it has proven disadvantageous that the shape of the wire structure is predetermined by the original shape of the shape memory material. Adaptation of the degree of expansion of the structure within the urethra is not possible. Thus, the expansion cannot be adapted in an optimized way individually in accordance with the anatomy of the patient. Rather, the same shape of implant is used for all patients. In addition, the correct positioning of the implant after expansion or after the unfolding of the structure is difficult or impossible since the implant has already anchored itself in the tissue. Adaptation of the expansion, such as, for example, an increase in the expansion, during treatment is also not possible. It would be conceivable, for example, that the BPH symptoms could be treated even more efficiently by enlarging the wire structure during treatment. However, since the known implant is limited to the predetermined shape, such enlargement is not possible. Likewise, it is difficult to fold the unfolded implant again and remove it from the urethra after completion of the treatment.

Proceeding therefrom, the underlying object of the invention is that of providing an implant which can be used in a more efficient and gentle manner for the treatment of the urinary tract.

One way of achieving this object is described by the features of claim 1. Accordingly, it is envisaged that an implant, in particular a removable implant, has a pressure means structure, which is constructed from at least two pressure means. These pressure means are of rod-like design and exert a local ischemic pressure on the tissue of the urethra. An essential feature of the invention is that the pressure means are secured with their distal ends on a common distal connecting body and with their proximal ends on a common proximal connecting body. In this case, the distal connecting body is secured in a fixed manner on a tightening means, while the proximal connecting body is movably mounted on this tightening means. By virtue of the movable mounting of the proximal connecting body on the tightening means and the fixing of the distal connecting body, the distance between the two connecting bodies can be varied. Reducing the distance between the two connecting bodies bends the pressure means. Here, the distance between the connecting bodies is inversely proportional to the degree of bending of the pressure means. The maximum distance between the two connecting bodies corresponds approximately to the length of the rod-like pressure means. In this state of maximum distance between the connecting bodies, the pressure means are in a state of minimum mechanical stress. Thus, by varying the distance between the two connecting bodies, the pressure means structure can be adapted individually and in accordance with the anatomy of the patient. In this case, by varying the distance between the two connecting bodies, the pressure means structure is expanded just as far as is particularly advantageous for the treatment. After completion of the treatment, the expanded pressure means structure can be contracted again by increasing the distance between the connecting bodies, thereby making it possible to recover the implant in a particularly easy and gentle manner.

The invention preferably provides for the pressure means structure to have three, preferably four, five, six or more pressure means. It has been found that particularly efficient treatment can be achieved by using three pressure means. Sufficient enlargement of the urethra can be achieved by expanding three pressure means to form the pressure means structure. Although a larger number of wires would have the advantage that a larger amount of tissue could be treated, it is likewise the case that more pressure means would require more space, which is disadvantageous in particular for the minimally invasive technique.

In particular, it is envisaged that the pressure means are wires made from a spring steel, stainless steel wires, plastic rods, rods made from a biodegradable material, or rods made from a material having a shape memory, in particular Nitinol. It is essential for the pressure means that they are biocompatible and have a high degree of flexibility. In addition, the pressure means must be sufficiently stable. In this case, the pressure means are of rod-like design and generally have a round cross section. However, it is also possible for the pressure means to have a different cross section. Customary diameters are a few tenths of a millimeter up to 1 mm. The use of biodegradable materials offers the advantage that the structure dissolves completely after a few days or weeks and recovery of the implant is not necessary.

Furthermore, provision can be made, according to the invention, for the tightening means to be a thread, a wire, a stainless steel wire, a plastic rod, a thread or a rod made from a biodegradable material, a rod or a wire having a screw thread or a rod or a wire having latching projections or notches. The tightening means must be sufficiently stable to move the distal connecting body relative to the proximal connecting body, specifically against the mechanical tightening force of the pressure means. It may be advantageous, particularly for the relaxation of the implant, if the tightening means is dimensionally stable, that is to say is designed as a wire or rod.

The invention envisages that—and the tightening means is designed accordingly—the proximal connecting body can be fixed, latched, clamped, screwed, twisted, crimped or the like on the tightening means in order to keep the distance between the distal connecting body and the proximal connecting body constant. Advantageously, the proximal connecting body itself fixes itself on the tightening means when the latter is pulled through the connecting body and the tensile force declines. In this case, the tightening means is fixed in the connecting body and the distance between the distal and the proximal connecting body is maintained, as a result of which the pressure means structure is also fixed in its shape.

A further advantageous exemplary embodiment of the invention envisages that the proximal connecting body has a latching means, a latching projection, a latching nose, a wedge, a closure or the like, in order to enter into a connection, in particular a releasable connection, with the tightening means. A further exemplary embodiment envisages that the proximal connecting body has a bore, a bore having an internal thread, a notch, a guide or a gap, through which the tightening means can be passed. It is also conceivable for the abovementioned latching means and the like to be arranged within the bore of the proximal connecting body. Thus, it is possible, for example, for a latching nose to be arranged in a bore or a passage in the connecting body, which latching nose, when the implant is tightened, engages against latching projections of the tightening means. When the implant has assumed the intended shape, the latching means in the connecting body prevents the pressure means from being relaxed. Another exemplary embodiment envisages that a screw thread is arranged in the connecting body, which screw thread can be moved by means of a screw connection or by a screwdriver on the tightening means, which can be designed as a threaded rod. In this exemplary embodiment, the internal thread of the connecting body prevents relaxation of the pressure means. If the screw thread or threaded rod is rotated in the opposite direction, the implant can be returned to its original shape. Another exemplary embodiment can provide for the tightening means to have small barbs which engage in corresponding receptacles in the connecting body and likewise counteract relaxation of the pressure means structure.

A particularly advantageous exemplary embodiment envisages that the latching means, the latching projection, the latching nose, the wedge, the closure or the like is produced from a biodegradable material. Using a biodegradable material for the latching mechanism ensures that the pressure means 12 relax automatically after the resorption period has elapsed. This eliminates the need for a further intervention, during which the latching mechanism must be released. The resorption process can be controlled very accurately by dimensioning the material and by selecting the composition of the material, with the result that the implant relaxes in a targeted manner at a specific time or in a specific time range. As a rule, this is after completion of the treatment.

The pressure means structure can be expanded by shortening the distance between the distal connecting body and the proximal connecting body on the tightening means, with the distances between the pressure means increasing. This increase in the distances between the pressure means can be carried out continuously or quasi-continuously. The distance between the pressure means can also be increased during treatment by reducing the distance between the connecting bodies. This is accomplished by pulling on the tightening means again or by a further rotation of the threaded rod or of the proximal connecting body having the internal thread. In the exemplary embodiment of the latching connection, the proximal connecting body is pulled further by a few latching projections. After completion of the treatment, this tightening process is carried out in the reverse order, with the result that the distance between the two connecting bodies is increased and the entire implant can be removed from the urethra.

Furthermore, a further exemplary embodiment of the invention can provide for each of the pressure means to have one, two or more inflection points about which the pressure means can be bent. It is also possible for an embodiment to provide for each of the pressure means to have two inflection points about which the pressure means bend when the connecting bodies are drawn together. As a result, the pressure means are subdivided into three sections, the central section running parallel to the tightening means and acting on the tissue of the urethra parallel to the other pressure means. By means of this multiple bending of the pressure means, the pressure which is exerted on the tissue by the pressure means can be intensified. This intensification of the pressure can lead to more efficient regression of the tissue.

A further embodiment of the invention envisages that two pressure means structures, namely a distal pressure means structure and a proximal pressure means structure, are arranged one behind the other on the tightening means, wherein the pressure means of the two pressure means structures are secured in a common central connecting body, and the distal pressure means structure has the distal connecting body and the proximal pressure means structure has the proximal connecting body. By means of these structures mounted one behind the other, the tissue can be treated particularly efficiently and in a manner adapted to the individual anatomy. By means of the two separate pressure means structures, it is possible to exert different pressures on the tissue along the urethra and thus to make the treatment particularly efficient.

The invention furthermore preferably envisages that the distal pressure means structure and the proximal pressure means structure each have a distal connecting body and a proximal connecting body, and both the distal pressure means structure and the proximal pressure means structure have a tightening means, thus enabling the pressure means structures to be tightened independently of one another. This possibility of expanding two pressure means structures arranged one behind the other individually and independently of one another offers the possibility of treating the urethra in a very targeted manner. It is thereby possible to react highly individually to various anatomies or BPH symptoms. In addition, the expansion of the pressure means structures can be readjusted independently of one another by virtue of this individual handling of the two pressure means structures, even during treatment. It is conceivable, depending on the course of treatment, that the tissue may denature at different rates at different points.

A preferred exemplary embodiment of the present invention is explained in greater detail below with reference to the drawing. In this drawing:

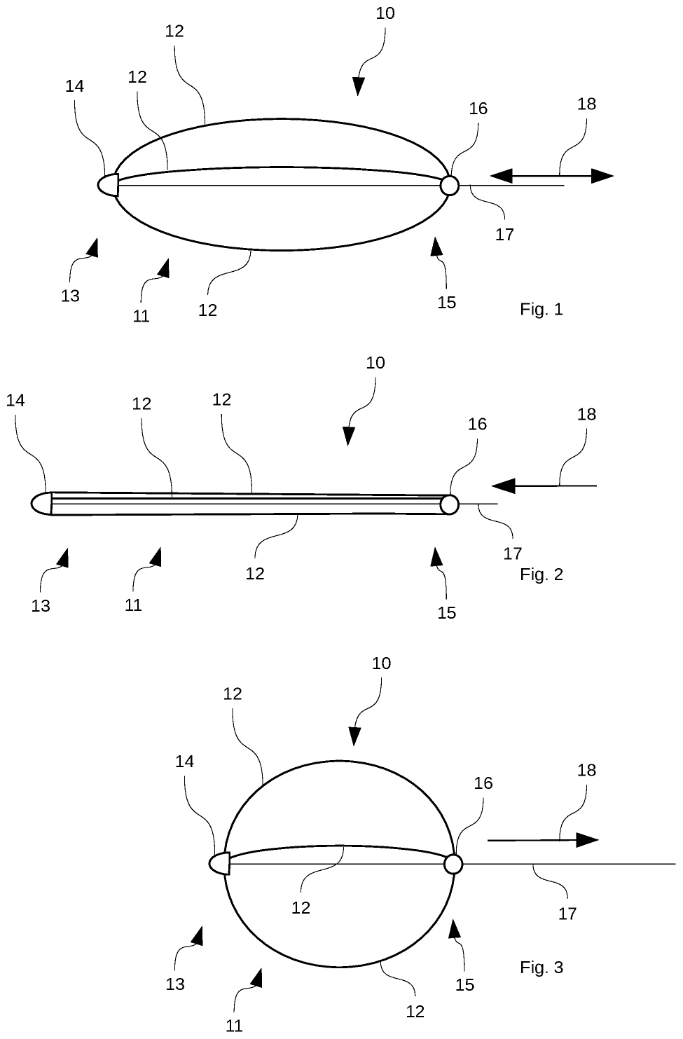

FIG. 1 shows a schematic illustration of an implant in a first position,

FIG. 2 shows a schematic illustration of the implant according to FIG. 1 in a second position,

FIG. 3 shows a schematic illustration of the implant according to FIG. 1 in a third position,

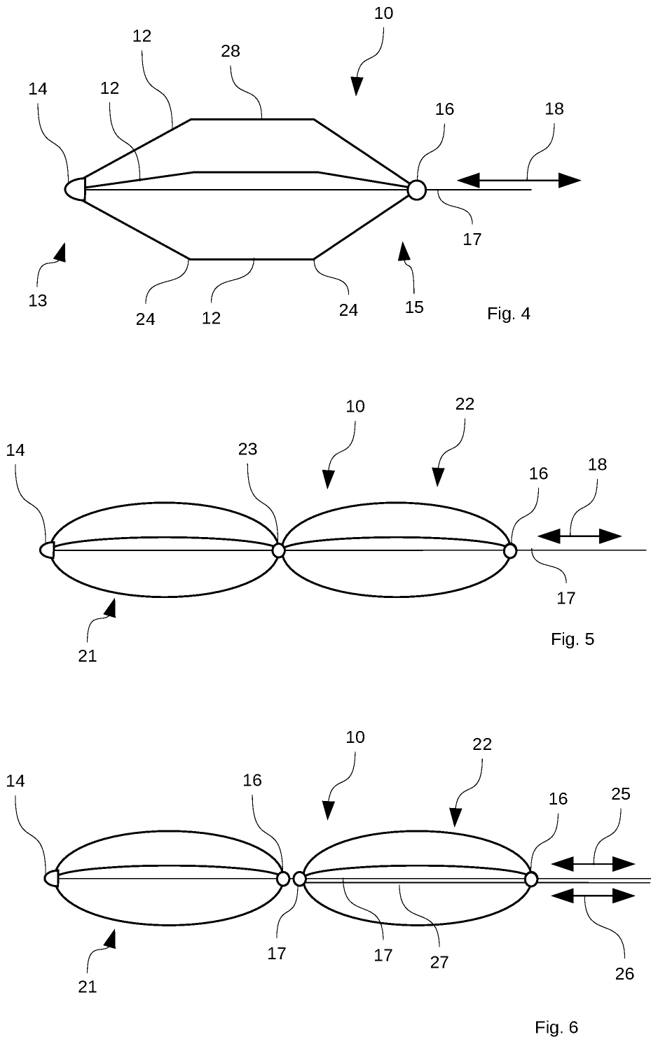

FIG. 4 shows a schematic illustration of a further exemplary embodiment of an implant,

FIG. 5 shows a schematic illustration of a further exemplary embodiment of an implant, and

FIG. 6 shows a schematic illustration of a further exemplary embodiment of an implant.

One possible exemplary embodiment of an implant 10 is illustrated in FIG. 1. It should be expressly pointed out that this example is only one of many conceivable embodiments. The invention envisages that all components can be designed in different ways.

The exemplary embodiment of the implant 10 which is illustrated in FIG. 1 has a pressure means structure 11 with three pressure means 12. These pressure means 12 are advantageously wires made from stainless steel, a spring steel or a material with a shape memory. Alternatively, it is also conceivable for the pressure means 12 to be plastic rods. Thus, in a particularly advantageous exemplary embodiment, it is envisaged that the plastic is biodegradable. The implant 10 thus dissolves at least partially after some time in the body, and therefore further intervention to recover the implant 10 is not necessary. In addition to the exemplary embodiment illustrated here, it is also conceivable for the pressure means structure 11 to have more than three pressure means 12. However, it has been found that three pressure means 12 are particularly suitable for manipulating the tissue of the urethra.

According to the invention, the distal ends 13 of the pressure means 12 are connected to a common distal connecting body 14. This distal connecting body 14 has a rounded portion on the distal side, thus enabling the implant 10 to be introduced into the human body without serious traumatization. The distal ends 13 of the pressure means 12 are inserted, glued or cast into the connecting body 14.

The opposite proximal ends 15 of the pressure means 12 are connected to a common proximal connecting body 16. In the figures, this proximal connecting body 16 is shown as a ball, but can also have any other shape. The proximal ends 15 of the pressure means 12 are connected to the proximal connecting body 16 in the same way as the distal ends 13 of the pressure means 12 are connected to the distal connecting body 14.

The distal connecting body 14 is also connected in a fixed manner to a tightening means 17. This tightening means 17 can be designed as a thread or as a wire. It is also conceivable for the tightening means 17 to be designed as a rod or pin. According to the exemplary embodiment illustrated in FIG. 1, the tightening means 17 of the implant 10 is passed through the proximal connecting body 15. However, it is also conceivable for the tightening means 17 to be guided along the proximal connecting body 16. In the figures, only a section of the tightening means 17 is shown. The tightening means 17 ending at the right-hand edge is substantially longer, thus enabling it also to be passed out of the body. According to the invention, the tightening means 17, which is designed as a thread or wire, can be moved back and forth along the double arrow 18. By pulling on the tightening means 17 in the proximal direction, the distal connecting body 14 is pulled in the direction of the proximal connecting body 16, the distance between the two connecting bodies 14, 16 being reduced (FIG. 3). To reduce the distance between the connecting bodies 14, 16, it is necessary to work against the mechanical stress energy of the pressure means 12. As can be seen in FIG. 3, but also already in FIG. 1, the distance between the two connecting bodies 14, 16 is inversely proportional to the bending of the pressure means 12. While, in FIG. 1, the pressure means 12 are only slightly bent or curved, the bending of the pressure means 12 in FIG. 3 can be described as very pronounced. By pulling further on the tightening means 17 in the direction of the arrow 19, the pressure means 12 can be bent even further.

In this stressed state of the implant 10, a spring force or restoring force acts continuously on the two connecting bodies 14, 16. Without a corresponding counterforce, the distal connecting body 14 is pressed in the direction of the arrow 20 by the proximal connecting body 16 (FIG. 2). In addition, a pressure force can be exerted on the distal connecting body 14 in the direction of the arrow 20 via the tightening means 17, with the result that the pressure means 12 are aligned almost completely straight and parallel to one another. In this state, the implant 10 has the smallest cross section.

For the treatment of BPH syndromes, the implant 10 is introduced into the body of the patient in the state illustrated in FIG. 2. As soon as the implant 10 is correctly positioned, the wire structure 11 is expanded by exerting a mechanical pull on the tightening means 17 in the direction of the arrow 19. To ensure, in particular, that the proximal connecting body 16 remains in its position, it is conceivable for the implant 10 to have an anchor structure (not illustrated), by means of which the implant 10 or the proximal connecting body 16 is held in its position. Should the implant 10 move out of the optimum position while the pressure means structure 11 is being expanded, the implant 10 can be moved back into the correct position.

As soon as the pressure means structure 11 has reached the optimum shape or expansion for the treatment of the tissue, the tightening means 17 is fixed by the proximal connecting body 16. For this purpose, the tightening means 17 is pulled a short distance in the distal direction on account of the spring stress of the pressure means 12, in order to latch immediately within the proximal connecting body. For this purpose, the proximal connecting body 16 can have corresponding latching means, barbs, hooks or the like. These latching means engage directly in the tightening means 17. It is envisaged according to the invention that the tightening means 17 has corresponding or complementary projections, notches or the like. Further expansion of the pressure means structure 11 in the direction of the arrow 19 is possible as before. To relax the pressure means structure 11, the latches must be released, or the tightening means 17 must be severed between the two connecting bodies 14, 16.

In the exemplary embodiment illustrated in FIG. 4, the pressure means 12 can be tightened not in the form of an arc but in the form of a trapezoid. For this purpose, the pressure means 12 each have two inflection points 24. It is conceivable in this exemplary embodiment for the pressure means 12 to be designed as rods made from plastic, which have a notch or a taper or weakening, which serve as inflection points 24. This exemplary embodiment proves to be particularly advantageous since, as a result of the expansion of the pressure means structure 11, straight flanks 28 of the pressure means 12, which are aligned parallel to the tightening means 17, are pressed against the tissue. As a result, it is possible to produce a channel-like passage through the urethra in a particularly efficient way.

FIG. 5 schematically illustrates a further exemplary embodiment of the implant 10 according to the invention. In this case, it is envisaged that two pressure means structures, namely a distal pressure means structure 21 and a proximal pressure means structure 22, are arranged one behind the other on the tightening means 17. These combined pressure means structures 21, 22 function in the same way as described in relation to the exemplary embodiment according to FIGS. 1 to 3. The tightening means 17 is connected to the distal connecting body 14 and can be latched in the proximal connecting body 16. In addition, the tightening means 17 runs through a central connecting body 23, on which the proximal and distal ends of the pressure means structures 21, 22, respectively, are also secured. By pulling on the tightening means 17 in the direction of the arrow 18, this double structure can be expanded or relaxed. By virtue of the two pressure means structures 21, 22, the tissue can be treated in a very targeted manner and over a larger section.

FIG. 6 illustrates a similar exemplary embodiment of the implant 10 to that in FIG. 5. However, in this exemplary embodiment, both the distal pressure means structure 21 and the proximal pressure means structure 22 have a distal connecting body 14 and a proximal connecting body 16. In addition, the two pressure means structures 21 and 22 each have a tightening means 17, 27, by means of which the pressure means structures 21, 22 can be expanded and relaxed independently of one another. By means of this targeted expansion or relaxation of two pressure means structures 21, 22, the treatment of the urethra can take place in different ways at different points. Thus, it is conceivable, for example, for the distal pressure means structure 21 to be expanded to a greater extent, using the tightening means 17, than the proximal pressure means structure 22, using the tightening means 27. Both pressure means structures 22, 23 can be moved independently of one another in the direction of the arrows 25, 26. Likewise, it is of course conceivable for more than two pressure means structures to be positioned one behind the other on tightening means in order to be able to treat the urethra in a targeted manner over a longer length.

### LIST OF REFERENCE SIGNS

10 implant
11 pressure means structure
12 pressure means
13 distal end
14 distal connecting body
15 proximal end
16 proximal connecting body
17 tightening means
18 double arrow
19 direction of arrow
20 direction of arrow
21 distal pressure means structure
22 proximal pressure means structure
23 central connecting body
24 inflection point
25 direction of arrow
26 direction of arrow
27 tightening means
28 flank

1. An implant for widening a urethra of a person by applying a local ischemic pressure to the tissue of the urethra, having a pressure means structure, which has at least two pressure means, wherein the implant can be introduced into the urethra with a distal end leading, wherein the pressure means are secured with their distal ends on a common distal connecting body and with their proximal ends on a common proximal connecting body, wherein the distal connecting body is secured in a fixed manner to a tightening means and the proximal connecting body is movably mounted on the tightening means.

2. The implant as claimed in claim 1, wherein the pressure means structure has three pressure means.

3. The implant as claimed in claim 1, wherein the pressure means are wires, wires made from a spring steel, stainless steel wires, plastic rods, rods made from a biodegradable material, or rods made from a material having a shape memory.

4. The implant as claimed in claim 1, wherein the tightening means is a thread, a wire, a stainless steel wire, a plastic rod, a thread or a rod made from a biodegradable material, a rod or a wire having a screw thread or a rod or a wire having latching projections or notches.

5. The implant as claimed in claim 1, wherein the proximal connecting body can be fixed, latched, clamped, screwed, twisted, crimped or the like on the tightening means in order to keep the distance between the distal connecting body and the proximal connecting body constant.

6. The implant as claimed in claim 1, wherein the proximal connecting body has a latching means, a latching projection, a latching nose, a wedge, a closure or the like, in order to enter into a connection, in particular a releasable connection, connection with the tightening means.

7. The implant as claimed in claim 6, wherein the latching means, the latching projection, the latching nose, the wedge, the closure or the like is produced from a biodegradable material.

8. The implant as claimed in claim 1, wherein the proximal connecting body has a bore, a bore having an internal thread, a notch, a guide or a gap, through which the tightening means can be passed.

9. The implant as claimed in claim 1, wherein the pressure means structure can be expanded by shortening the distance between the distal connecting body and the proximal connecting body on the tightening means, with the distances between the pressure means increasing.

10. The implant as claimed in claim 1, wherein the pressure means each have one, two or more inflection points about which the pressure means can be bent.

11. The implant as claimed in claim 1, wherein two pressure means structures, namely a distal pressure means structure and a proximal pressure means structure, are arranged one behind the other on the tightening means, wherein the pressure means of the two pressure means structures are secured in a common central connecting body, and the distal pressure means structure has the distal connecting body and the proximal pressure means structure has the proximal connecting body.

12. The implant as claimed in claim 11, wherein the distal pressure means structure and the proximal pressure means structure each have a distal connecting body and a proximal connecting body, and both the distal pressure means structure and the proximal pressure means structure have a tightening means, thus enabling the pressure means structures to be tightened independently of one another in accordance with the directions of the arrows.
